(19) United States
(12) Patent Application Publication
(10) Pub. No.: US 2005/0096217 A1
(43) Pub. Date: May 5, 2005

(54) Selective hydrogenation catalyst

(75) Inventors: Andrezej Rokicki, Prospect, KY (US); Jennifer A. Boyer, Jeffersonville, IN (US); Steven A. Blankenship, Radcliff, KY (US)

Correspondence Address: Scott R. Cox;LYNCH, COX, GILMAN & MAHAN, PSC, Ste. 2200, 400 West Market Street, Louisville, KY, 40202, US

(73) Assignee: SUD-CHEMIE, INC., LOUISVILLE, KY (US)

(21) Appl. No.: 10/696,749

(22) Filed: Oct. 29, 2003

(51) Int. Cl. C07C 5/03; C07C 5/08; C07C 7/163; C07C 7/167

(52) U.S. Cl. 502/327; 585/259; 502/339; 502/333

(57) ABSTRACT

A process for selective hydrogenation of acetylene during ethylene purification utilizing a palladium/thallium impregnated catalyst.

### BACKGROUND OF INVENTION

This invention relates to a catalyst useful for selective hydrogenation of unsaturated compounds, such as acetylene, in an olefinic feed stream, particularly for front-end ethylene purification. This invention also relates to a process for the preparation of the catalyst and the use of the catalyst for the selective hydrogenation of unsaturated compounds, such as acetylene, particularly for front-end ethylene purification.

The manufacture of unsaturated hydrocarbons usually involves cracking various types of hydrocarbons and often produces a crude product containing hydrocarbon impurities that are more unsaturated than the desired product. These unsaturated hydrocarbon impurities are often very difficult to separate by fractionation from the desired product. An example of this problem occurs with ethylene purification, in which acetylene is a common impurity. It is often difficult, industrially, to remove undesirable, highly unsaturated hydrocarbons by hydrogenation without significant hydrogenation of the desired hydrocarbons.

Two general types of gas phase selective hydrogenation processes for removing undesired, unsaturated hydrocarbons are commonly used. One type, known as “front-end” hydrogenation, involves passing the crude gas from the initial cracking step, after removal of steam and condensible organic material, over a hydrogenation catalyst. The crude gas has a large hydrogen content relative to the quantity of acetylenes present, thereby, theoretically making possible the hydrogenation of all of those acetylenes as well as a substantial quantity of the ethylene that is present. In practice, substantially complete hydrogenation of the acetylenes with sufficient selectivity to produce olefins of polymerization quality is often a problem. The high concentration of hydrogen present in the front-end systems results in the need for a very selective catalyst that does not also substantially hydrogenate desirable components of the feed stream, such as ethylene. Overhydrogenation can lead to a thermal excursion in reactors, which is also known as “run-away”. Under “run-away” conditions, excessively high temperatures are experienced, severe loss of ethylene occurs, and catalyst damage takes place. Another problem that can occur in a front-end reactor system is a furnace upset which can result in swings in the CO concentration from moderate levels to very low levels. Conventional, front-end catalysts cannot tolerate these large swings in CO concentration very well which often produce “run-away” conditions.

In the other type of gas phase selective hydrogenation, which is known as “tail-end” hydrogenation, the crude gas is first fractionated and the resulting concentrated product streams are individually reacted with hydrogen in a slight excess over the quantity required for hydrogenation of the undesirable, highly unsaturated hydrocarbons, such as acetylene. However, in tail-end processes there is a greater tendency for deactivation of the catalyst, and consequently, periodic regeneration is necessary. While thermal excursion is not a concern, formation of undesirable polymers is often a major problem.

A number of patents have discussed selective hydrogenation of unsaturated hydrocarbons, such as U.S. Pat. Nos. 4,126,645, 4,367,353, 4,329,530, 4,347,392 and 5,414,170. The catalysts that are preferred for selective hydrogenation reactions conventionally utilize palladium, supported on an alumina substrate, as disclosed, for example, in U.S. Pat. Nos. 3,113,980, 4,126,645 and 4,329,530. Other gas phase, palladium on alumina catalysts for the selective hydrogenation of acetylene compounds are disclosed, for example, in U.S. Pat. Nos. 5,925,799, 5,889,138 and 5,648,576.

One of the problems with conventional palladium on alumina catalysts is that under normal operating conditions not only is the acetylene hydrogenated, a substantial proportion of the ethylene is also converted to ethane. In addition, the palladium on alumina catalysts often have relatively low stability due to the formation of large quantities of oligomers on the catalyst surface during the selective hydrogenation process.

To overcome these problems, promoters or enhancers are added to the catalyst. One common enhancer for a conventional palladium on alumina selective hydrogenation catalysts is silver. Acetylene hydrogenation catalysts for ethylene purification comprising palladium and silver on a support material are disclosed in U.S. Pat. Nos. 4,404,124, 4,484,015, 5,488,024, 5,489,565 and 5,648,576.

Catalysts comprising palladium, silver, an alkali metal fluoride and a support material, which are utilized for the hydrogenation of feed stream impurities, such as dienes and diolefins, are disclosed, for example, in U.S. Pat. No. 5,489,565.

Catalysts useful for hydrogenation of organic compounds are also disclosed in U.S. Pat. Nos. 6,255,548 and 6,294,696 and comprise at least one support, at least one metal from Group VIII, and at least one additional element, M, selected from the group consisting of germanium, tin, lead, rhenium, gallium, indium, gold, silver and thallium, preferably tin or germanium. The catalyst for this process is prepared by introducing a metal into an aqueous solvent, preferable in the form of an organometallic compound containing at least one carbon-M bond. See also U.S. Pat. Nos. 3,962,139 and 6,225,516.

U.S. Pat. No. 6,465,391 discloses a selective hydrogenation catalyst and processes for the production thereof, wherein the catalyst comprises an inorganic support material, a palladium component, a silver component, and a promoter component having the formula XYFn, where an X is an alkaline metal, Y is an element selected from the group consisting of antimony, phosphorous, boron, gallium, aluminum, indium, thallium, and arsenic and n is an integer which makes YFn an monovalent anion.

U.S. Pat. No. 3,992,468 discloses a process for hydrodealkylating alkylaromatic hydrocarbons using a catalyst comprising two metals, the first of the metals selected from the group consisting of cobalt, ruthenium, osmium, palladium, rhodium, indium, and platinum or from the group consisting of molybdenum, tungsten, and manganese, and the second metal selected from the group consisting of zinc, cadmium, gallium, indium, thallium, manganese, copper, silver, gold, yttrium, titanium, niobium, tantalum, chromium, molybdenum, tungsten, rhenium, germanium, tin and lead.

While conventional silver/palladium-based catalysts for the selective hydrogenation of acetylene have been useful, there are a number of problems that have been discovered from their use, including relatively low tolerance to carbon monoxide concentration swings and lower selectivity than is desirable by the industry.

The catalysts of the invention are designed to address these deficiencies in conventional ethylene purification catalysts.

Accordingly, it is an object of this invention to disclose a process for the selective hydrogenation of an olefinic feed stream containing acetylenic impurities, particularly for ethylene purification.

It is a further object of this invention to disclose a process for front-end selective hydrogenation of acetylenic impurities, whereby the quantity of the desirable olefins, particularly ethylene, is not substantially reduced.

It is a further object of the invention to disclose a palladium/thallium catalyst for use in the selective hydrogenation of acetylenic impurities, particularly for use in front-end ethylene purification.

It is a further object of the invention to disclose a palladium/thallium catalyst for selective hydrogenation of acetylenic impurities which contains precise quantities of palladium and thallium.

It is a still further object of the invention to disclose a palladium/thallium selective hydrogenation catalyst for the selective hydrogenation of acetylene which exhibits improved selectivity, resistance to run-away, and tolerance to CO concentration swings in comparison with conventional palladium or palladium/silver selective hydrogenation catalysts.

These and other objects can be obtained by the disclosed selective hydrogenation catalyst and process for the preparation and use of the selective hydrogenation catalyst for use in an olefinic feed stream containing acetylenic impurities, particularly for ethylene purification.

### SUMMARY OF THE INVENTION

The present invention is a process for the production and distribution of a catalyst for the selective hydrogenation of acetylenic impurities for ethylene purification comprising
preparing a carrier material in a suitable shape;
impregnating the carrier with a palladium metal source, preferably in solution;
calcining the palladium-impregnated carrier;
impregnating the palladium-impregnated carrier with a thallium-metal source, preferably in solution,
calcining the palladium/thallium impregnated carrier; and
reducing the palladium and thallium materials, wherein the quantity of the reduced palladium, by weight, including the palladium, comprises from about 0.001 to about 2 weight percent, the quantity of the reduced thallium, by weight including the thallium, comprises from about 0.001 to about 1 weight percent and wherein the concentration of the palladium metal is not less than the concentration of the thallium metal.

Preferably the reduced catalyst is then sealed into shipping containers under a non-oxidizing material for shipment.

The present invention further comprises a palladium/thallium catalyst for front-end selective hydrogenation of acetylenic impurities comprising from about 0.001 to about 2 weight percent palladium, including the palladium, and from about 0.001 to about 1 weight percent thallium, including the thallium, on a low surface area carrier i.e. less than 100 m²/g, wherein the concentration of the palladium metal is not less than the concentration of the thallium metal. Preferably the ratio of the palladium metal to the thallium metal is 1:1 or less, more preferably about 10:1 or less.

The invention further comprises a process for the selective hydrogenation of acetylenic impurities for front-end ethylene purification comprising passing an ethylene feed stream containing acetylenic impurities over the catalyst described above.

### DETAILED DESCRIPTION

The catalyst of the invention is designed primarily for the selective hydrogenation of acetylene in admixture with ethylene, particularly for front-end processes. The feed stream for this selective hydrogenation process normally includes substantial quantities of hydrogen, methane, ethane, ethylene, small quantities of carbon monoxide and carbon dioxide, as well as various impurities, such as acetylene. The goal of the selective hydrogenation process is to reduce substantially the amount of the acetylenic impurities present in the feed stream without substantially reducing the amount of ethylene that is present. If substantial hydrogenation of the ethylene occurs, thermal run-away can occur which adversely affects the catalyst. The catalyst of the invention exhibits enhanced selectivity, resistance to run-away, and better tolerance for CO concentration swings than is experienced using conventional selective hydrogenation catalysts.

The catalyst that is useful for this selective hydrogenation process is comprised of a low surface area carrier into which palladium and thallium are impregnated. The catalyst carrier is any relatively low surface area catalyst carrier manufactured from alumina, alpha alumina, zinc oxide, nickel spinel, titania, magnesium oxide, cerium oxide and mixtures thereof. In a preferred embodiment, the catalyst carrier is an alpha alumina. The surface area of the catalyst carrier is preferably from about 1 to about 100 m²/g, more preferably from about 1 to about 50 m²/g, and most preferably from about 1 to about 10 m²/g. Its pore volume is from about 0.2 to about 0.7 cc/g, preferably from about 0.3 to about 0.5 cc/g.

The catalyst carrier can be formed in any suitable size and in any suitable shape, such as spherical, cylindrical, trihole trilobal, monolith, pellet, tablet, ring and the like. In a preferred embodiment the catalyst carrier is formed in a tablet shape with a diameter from about 3 to about 5 mm.

Palladium can be introduced into the catalyst carrier by any conventional procedure. The presently preferred technique involves impregnating the catalyst carrier with a palladium metal source, preferably in the form of an aqueous solution of a palladium salt, such as palladium chloride or palladium nitrate, preferably palladium chloride. The extent of penetration of the palladium salt is preferably controlled by adjustment of the pH of the solution. In a preferred embodiment, the depth of penetration of the palladium salt is controlled such that approximately 90 percent of the palladium salt is contained within about 250 microns of the surface of the catalyst carrier. Any suitable method can be used to determine palladium penetration, such as is disclosed in U.S. Pat. Nos. 4,484,015 and 4,404,124. After palladium impregnation, the intermediate impregnated catalyst composition is calcined at a temperature from about 400° C. to about 600° C. for at least one hour.

Once the palladium-impregnated intermediate catalyst composition has been calcined, that composition is further impregnated with a thallium metal source, preferably a thallium metal solution such as HCO₂Tl. The palladium/thallium impregnated catalyst material is then calcined at a temperature from about 400° C. to about 600° C. for at least one hour.

In an alternative process of manufacture, the thallium and palladium metals can be co-impregnated and then calcined. Notwithstanding, it is preferable that thallium metal source not be introduced before the source for the palladium metal.

While the reduction process can occur in situ within a front-end reactor, the metals of the catalyst are preferably reduced in a reduction furnace prior to shipment. The metal compounds contained in the thallium/palladium catalyst precursor are preferably reduced by heating the catalyst while under a reducing gas, at a temperature from about 94° C.-535° C., preferably from about 94° C.-260° C. for a time sufficient to reduce the palladium and thallium metal sources. Preferable reducing gases include hydrogen, carbon monoxide and mixtures thereof. The catalyst is then cooled under a purge gas, such as nitrogen, to room temperature. Other conventional reduction processes may alternatively be used.

The amount of palladium present after reduction is from about 0.001 to about 2 weight percent, preferably from about 0.005 to about 0.05 weight percent, and most preferably from about 0.01 to about 0.03 weight percent based on the total weight of the catalyst, including the palladium. The amount of thallium present in the catalyst after reduction is from about 0.001 to about 1 percent, preferably 0.001 to 0.03 weight percent, and most preferably from about 0.001 to about 0.01 weight percent, based on the total weight of the catalyst, including the thallium.

It has been discovered that a useful catalyst is produced when the concentration of palladium metal equals or exceeds the concentration of thallium metal with a preferable Pd:Tl ratio from 1:1 to about 100:1, a more preferable Pd:Tl ratio from 5:1 to about 50:1, and a most preferable Pd:Tl ratio from about 10:1 to about 20:1, calculated as metals.

Following a final drying step, the thallium/palladium containing catalyst is prepared for shipment. The catalyst is preferably loaded into individual containers under a non-oxidizing gaseous atmosphere for shipping. Preferable non-oxidizing gases include nitrogen, argon, carbon dioxide or mixtures thereof.

In use, the catalyst is placed in the bed of a reactor. If desired the catalyst can be reduced in situ as is possible in some operations. Alternatively, the catalyst, which has been reduced prior to shipment, is merely placed within a catalyst bed ready for use. Selective hydrogenation of acetylene occurs when a gas stream containing primarily hydrogen, ethylene, methane, unsaturated impurities, such as acetylene, and minor amounts of carbon monoxide is passed over the catalyst of the invention. The inlet temperature of the feed stream is raised to a level sufficient to hydrogenate the acetylene. Generally, this temperature range is about 35° C. to about 100° C. Any suitable reaction pressure can be used. Generally, the total pressure is in the range of about 100 to 1000 psig with the gas hourly space velocity (GHSV) in the range of about 1000 to about 14000 liters per liter of catalyst per hour.

Conventional palladium/silver catalysts are often prone to run-away conditions, especially when the quantity of carbon monoxide changes dramatically during the reaction process. The catalyst of the invention is resistant to these run away conditions even when the quantity of carbon monoxide is low. Further, the catalyst of the invention exhibits enhanced selectivity over prior art catalysts. By the process of use of the catalyst of the invention, reduction of acetylene to a level less than about 1 ppm can be achieved.

Regeneration of the catalyst may be accomplished by heating the catalyst in air at a temperature, preferably not in excess of 500° C., to burn off any organic material, polymers or char.

### EXAMPLES

### Example 1

### Comparative

A commercially available, palladium/alumina catalyst manufactured by Sud-Chemie Inc. under the product name G-83A is obtained. Analysis shows that the catalyst comprises a palladium on alumina catalyst containing 0.018 weight percent palladium. The carrier is comprised of 99 weight percent alumina. The catalyst has a BET surface area of 3.7 m²/g.

### Example 2

### Comparative

A commercially available catalyst manufactured by Sud-Chemie Inc. under the product name of G-83C is obtained. Analysis shows that the catalyst comprises a palladium/silver on alumina catalyst containing 0.018 weight percent of palladium and 0.07 weight percent of silver on 99 weight percent alumina. The catalyst has a BET surface area of about 4.3 m²/g.

### Example 3

A catalyst is prepared by dipping 100 grams of a commercially available, low surface area alumina spheres with a BET surface area of 50 m²/g in a PdCl₂ solution to yield a palladium loading of 0.03 weight percent, including the palladium, with a palladium depth of penetration that is controlled to wherein at least about 90 percent of the palladium is within 250 microns of the surface of the spheres. After palladium impregnation, the intermediate catalyst is calcined at 454° C. for 3 hours. The palladium-containing intermediate is then impregnated with thallium in the form of HCO₂Tl to yield a thallium loading of 0.03 weight percent, including the thallium. The weight ratio between the palladium metal and the thallium metal on a by weight basis is 1:1. The catalyst containing the palladium and thallium is calcined a second time at about 454° C. for 3 hours. The catalyst is then loaded into a reduction bed, and purged with nitrogen while the bed is heated to 94° C. Once this temperature is reached, the nitrogen purge gas is discontinued and hydrogen gas is introduced as a reducing gas. The bed is maintained at 94° C. for at least 60 minutes. Upon completion of the reduction cycle, nitrogen gas is reintroduced into the bed and the bed is cooled to room temperature.

### Example 4

A catalyst is prepared according to Example 3 except the low surface area alumina has a BET surface area of 5 m²/g. The catalyst is then loaded into a reduction bed, purged with nitrogen while the bed is heated to 94° C., and reduced as described in Example 3.

### Example 5

A catalyst is prepared according to Example 3 except the weight ratio between the palladium metal and thallium metal was 10:1 Pd:Tl (0.03 weight percent palladium, including the palladium, and 0.003 weight percent thallium, including the thallium). Further, the catalyst is not reduced.

### Example 6

The catalyst from Example 5 is reduced as described in Example 3.

### Example 7

A catalyst is prepared according to Example 6 except that the low surface area alumina has a BET surface area of 5 m²/g.

Tables

Performance Testing:

Table 1, which follows, provides a comparison of the performance of Examples 1 and 2 (Comparative Examples) with Examples 3 through 7. The samples are compared by passing a conventional ethylene feed stream over the catalysts. The catalysts are evaluated in a bench scale laboratory, one-half inch i.d. reactor tube, which simulated a front-end feed stock reactor.

Catalyst activity and selectivity are evaluated. For each catalyst, the inlet temperature is recorded when less than 25 ppm acetylene leakage is detected at the reactor outlet. This temperature, T₁, is designated as the lower reaction temperature for catalyst activity. The inlet temperature is then increased until “run-away” is observed. “Run-away” or thermal excursion is defined as a greater than 4 percent H₂ loss in the system, and occurs when the hydrogenation of ethylene (C₂H₄) is significant. The temperature of the reactor inlet when run-away is noted is reported as T₂. The catalyst activity then is evaluated in terms of the temperature range over which the catalyst could effectively function, or the temperature at which hydrogenation is first observed (T₁) to the temperature at which run-away occurs (T₂). A large delta T (T₂-T₁) indicates that the catalyst can operate effectively over a broad temperature range. As the reactor temperature is increased, the hydrogenation reaction becomes more active with a greater amount of C₂H₂ being hydrogenated and hence, removed from the product stream. However, some hydrogenation of C₂H₄ also occurs indicating a loss of selectivity for the reaction. As shown in Table I, “selectivity” of each catalyst is reported as a percentage and is determined by the following calculations: 100 times (inlet C₂H₂— outlet C₂H₂) minus (C₂H₆ outlet minus C₂H₆ inlet)/(C₂H₂ inlet minus C₂H₂ outlet) times 100. Higher positive percentages indicate a more selective catalyst. Data was obtained at a moderate GHSV (7000).

TABLE I
Table I - 7000 GHSV activity/selectivity test

| | | | | Activity | |
|---|---|---|---|---|---|
| | | T₁ | T₂ | Range | Selectivity |
| Run | Catalyst | (° C.) | (° C.) | T₂-T1 | at T₁ |
| Comparative | (G83A) Pd/Al₂O₃ | 60 | 66 | 6 | +3% |
| Example 1 | no pre-reduction | | | | |
| Comparative | (G83C) Pd/Ag/Al₂O₃ | 46 | 52 | 6 | −125% |
| Example 2 | no pre-reduction | | | | |
| Example 3 | 1:1 Pd:Tl (0.03% Pd, | 46 | 58 | 12 | −41.8% |
| | O.03% Tl) on alumina | | | | |
| | 50 m²/g S.A. | | | | |
| | (reduced) | | | | |
| Example 4 | 1:1 Pd:Tl (0.03% Pd, | 37 | 57 | 20 | +34% |
| | O.03% Tl) on alumina | | | | |
| | 5 m²/g S.A. (reduced) | | | | |
| Example 5 | 10:1 Pd:Tl (0.03% Pd, | 44 | 58 | 14 | +46.6% |
| | 0.003% Tl) on alumina | | | | |
| | (50 m²/g S.A.) (No | | | | |
| | pre-reduction) | | | | |
| Example 6 | 10:1 Pd:Tl (0.03% Pd, | 67 | 84 | 17 | +79% |
| | O.003% Tl) on | | | | |
| | alumina (50 m²/g | | | | |
| | S.A.) (reduced) | | | | |
| Example 7 | 10:1 Pd:Tl (0.03% Pd, | 58 | 77 | 19 | +41.7% |
| | 0.003% Tl) on alumina | | | | |
| | (5 m²/g S.A.) | | | | |
| | (reduced) | | | | |

Comparison of the activity range and the selectivity for the prior art catalysts (Examples 1-2) to the inventive catalysts (Examples 3-7) demonstrates the enhanced performance of the catalysts of the invention. Selectivity is significantly improved relative to the prior art catalysts. Further, the catalysts of the invention demonstrate a broader temperature range over which the catalysts are active for hydrogenation than the prior art catalysts.

CO Concentration Swings

Feedstreams supplied to commercial front-end hydrogenation reactors can have substantial swings in CO concentration. This occurs when a new hydrocarbon cracker is brought on-line. The CO present in the feedstream acts as a selectivity enhancer. If the quantity of CO drops dramatically, thermal excursion can occur with existing commercial catalyst. To predict the performance of the catalysts of the invention under this condition, a test was developed to mimic CO concentration swings which often occur in ethylene plants. Selected catalysts are tested under CO swing test conditions. The feed consists of 0.25% C₂H₂, 20% H₂, 247 ppm CO, 45% C₂H₄ and 34% CH₄. The temperature was increased until the reactor exit C₂H₂ levels reached 97% conversion. The CO level was then reduced by a mass flow controller to 100 ppm. Test results are summarized in Table II.

TABLE II

| | | |
|---|---|---|
| | Comparative | |
| Catalyst | Example 2 | Example 7 |

| Pre-reduced | No | | Yes | |
|---|---|---|---|---|
| CO level(ppm) | 247 | 100 | 247 | 100 |
| Temperature to | 43 | | 44 | |
| reach ˜97% | | | | |
| conv. (° C.) | | | | |
| Conversion | 98.1 | Run-away | 96.3 | 99.6 |
| Selectivity @ | −5.7 | Run-away | 29.9 | −146.9 |
| 97% | | | | |
| Conversion | | | | |

The catalyst of Example 7 showed enhanced selectivity over the commercially available catalyst of Comparative Example 2. Thus the catalyst of the invention is more tolerant to CO reduction.

The principles, preferred embodiments, and modes of operation of the present invention have been described in the foregoing specification. The invention which is intended to be protected herein, however, is not to be construed or limited to the particular terms of disclosure, as these are to be regarded as being illustrative, rather than restrictive. Variations and changes may be made by those skilled in the art without departing from the invention.

1. A catalyst for the selective front-end hydrogenation of acetylene comprising
an inorganic support, a palladium metal source, and a thallium metal source, wherein the palladium metal source comprises from about 0.001 to about 2 weight percent, and the thallium metal source comprises from about 0.001 to about 1 weight percent, wherein the weight percentages are based on the total weight of the catalyst, including the palladium and thallium, and wherein the concentration of palladium metal is not less than the concentration of thallium metal.

2. The catalyst of claim 1 wherein at least about 90 percent of the palladium metal source is concentrated within about 250 microns of a surface of the catalyst.

3. The catalyst of claim 1 wherein the inorganic support is selected from the group consisting of alpha alumina, zinc oxide, nickel spinel and other low surface area catalyst support materials, and mixtures thereof, with a surface area less than about 100 m²/g.

4. The catalyst of claim 1 formed in the shape of a sphere, trihole trilobal, monolith, pellet, ring or tablet.

5. The catalyst of claim 1 wherein the support material has a BET surface area in the range of about 1 to about 100 m²/g.

6. The catalyst of claim 1 wherein the support material has a pore volume in the range of about 0.2 to about 0.7 cc/g.

7. The catalyst of claim 1 wherein the palladium metal comprises from about 0.005 to about 0.05 weight percent of the catalyst, based on the total weight of the catalyst, including the palladium metal.

8. The catalyst of claim 1 wherein the palladium metal comprises from about 0.01 to about 0.03 weight percent of the catalyst based on the total weight of the catalyst, including the palladium metal.

9. The catalyst of claim 1 wherein the thallium metal comprises from about 0.001 to about 0.01 weight percent of the catalyst based on the total weight of the catalyst, including the thallium metal.

10. The catalyst of claim 1 wherein the ratio of the palladium metal to the thallium metal is from 1:1 to about 100:1.

11. The catalyst of claim 1 wherein the ratio of the palladium metal to the thallium metal is from about 5:1 to about 50:1.

12. The catalyst of claim 1 wherein the ratio of the palladium metal to the thallium metal is from about 10:1 to about 20:1.

13. A process for the manufacture of a catalyst for the selective hydrogenation of acetylene comprising
preparing a low surface area catalyst support,
impregnating the catalyst support with a palladium metal source, wherein the palladium metal source is selected from the group consisting of palladium salt and metallic palladium, and
impregnating the palladium-impregnated catalyst support with a thallium metal source, wherein the thallium metal source is selected from the group consisting of a thallium salt and metallic thallium, wherein the concentration of the thallium metal does not exceed the concentration of the palladium metal.

14. The process of claim 13 wherein the depth of penetration of the palladium metal source into the catalyst support is wherein about 90 percent of the palladium is present within about 250 microns of the surface of the catalyst material.

15. The process of claim 13 wherein the ratio of the palladium metal to the thallium metal, calculated as elemental metals, is from 1:1 to about 100:1.

16. The process of claim 13 wherein the ratio of the palladium metal to the thallium metal calculated as elemental metals, is from about 5:1 to about 50:1.

17. The process of claim 13 wherein the ratio of the palladium metal to the thallium metal calculated as elemental metals, is from about 10:1 to about 20:1.

18. The process of claim 13 further comprising reducing the catalyst by heating the catalyst in a reducing furnace under a reducing gas.

19. The process of claim 18 wherein the reducing gas is selected from hydrogen, carbon monoxide or mixtures thereof.

20. A process for the selective acetylene hydrogenation in a front-end ethylene purification process comprising
preparing the palladium/thallium catalyst of claim 1,
passing a feed stream comprising methane, ethylene, hydrogen, carbon monoxide and acetylene over the catalyst.

21. The process of claim 21 wherein the amount of the acetylene contained in the feed stream is reduced to less than about 1 ppm.
